(19) United States
(12) Patent Application Publication
(10) Pub. No.: US 2007/0224230 A1
(43) Pub. Date: Sep. 27, 2007

(54) Cosmetic composition based on zinc and copper sulphates and sucralphate

(75) Inventors: Pierre Fabre, Castres (FR); Anne-Sophie Dussert, Toulouse (FR); Michel Jeanjean, Tolosan (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE DERMO-COSMETIQUE, BOULOGNE (FR)

(21) Appl. No.: 11/786,577

(22) Filed: Apr. 12, 2007

Related U.S. Application Data
(63) Continuation of application No. 10/477,259, filed on Apr. 2, 2004, now abandoned.

(30) Foreign Application Priority Data
May 11, 2001 (FR) FR01/06242

(51) Int. Cl. A61K 8/02; A61K 33/30; A61K 33/34

(52) U.S. Cl. 424/401; 424/637; 424/642

(57) ABSTRACT

The invention relates to a cosmetic composition comprising an association of sucralphate and a mixture of zinc and copper sulphates in an excipient suitable for topical application to the skin. More specifically, said composition is intended for the regenerating, healing and/or anti-inflammatory treatment of the skin.

The present invention relates to cosmetic formulations containing sucralfate in combination with copper sulfate and zinc sulfate, used as tissue regenerators, cicatrizing agents and antiinflammatory agents.

Sucralfate is basic aluminum sucrose sulfate, and is used as a medicinal product in the treatment of gastric and duodenal ulcers under the brand names Ulcar® and Keal®.

When absorbed at a dose of from 0.5 to 2 g per day in a dry form such as a tablet or chewable granules, sucralfate acts on the digestive tract by lining the mucous membrane of the stomach and the duodenum with a protective gel.

The formation of this gel is consecutive to the reaction that takes place between sucralfate and the hydrochloric acid of the gastric and duodenal medium, and, as a result of the electromagnetic tropism it displays toward positively charged protein molecules, it forms a complex with them that insulates and protects gastric ulcers.

Moreover, sucralfate inhibits the proteolytic activity of pepsin. Thus, it allows and promotes the natural cicatrization of ulcers.

The present invention relates to the cosmetic use, thus via the topical route, of formulations containing sucralfate in combination with copper sulfate and zinc sulfate, as tissue regenerators, cicatrizing agents and calmatives.

According to one particular characteristic of the present invention, the cosmetic composition has a sucralfate content of between 0.01% and 5% by weight and preferably about 1% by weight.

According to another characteristic of the present invention, the cosmetic composition comprises from 0.02% to 2% by weight and preferably between 0.3% and 2% by weight of sulfates.

According to another characteristic of the present invention, the composition comprises a weight ratio of sucralfate to sulfates of between 0.5 and 20 and preferably between 0.5 and 1.

According to another characteristic of the present invention, the cosmetic composition contains a weight ratio of copper sulfate to zinc sulfate of between 1 and 3.

The formulation examples given below are intended to illustrate the invention and are cited in a purely nonlimiting manner.

EXAMPLE 1
Water-in-oil cream

| | | |
|---|---|---|
| Avène spring water qs | 100 | g |
| Micronized sucralfate | 1 | g |
| Copper sulfate | 0.2 | g |
| Zinc sulfate | 0.1 | g |
| Zinc oxide | 4 | g |
| Glycerol | 5 | g |
| Hostacerin WO (polyglyceryl-2 sesquiisostearate + | 3.7 | g |
| beeswax + mineral oil + magnesium aluminum stearate) | | |
| Cremiol HF 52 (hydrogenated plant oil) | 5 | g |
| Liquid paraffin | 8 | g |
| Caprylic/capric triglyceride | 19 | g |
| Elfaros ST 37 (PEG 22 dodedcyl glycol copolymer) | 1.2 | g |
| Propylene glycol | 3 | g |
| Magnesium sulfate | 0.1 | g |

EXAMPLE 2
Emulsion 1 (oil-in-water)

| | | |
|---|---|---|
| Caprylic/capric triglyceride | 7 | g |
| Passionflower oil | 7 | g |
| Glyceryl stearate + stearyl alcohol + ceteth 20 + | 6.5 | g |
| steareth 25 | | |
| Shea butter | 3 | g |
| Dimethicone | 2 | g |
| Sodium Carbomer | 0.35 | g |
| Sucralfate | 0.01 | g |
| Copper sulfate | 0.01 | g |
| Zinc sulfate | 0.01 | g |
| Demineralized water qs | 100 | mg |

EXAMPLE 3
Emulsion 2 (O/W)

| Liquid paraffin | 10 | g |
|---|---|---|
| Caprylic/capric triglyceride | 7 | g |
| Cyclomethicone | 3 | g |
| Sucrose stearate | 2 | g |
| Sucrose distearate | 2 | g |
| Carbomer | 0.4 | g |
| Cakile | 2 | g |
| Triethanolamine qs | pH 7 | |
| Sucralfate | 5 | g |
| Zinc sulfate | 0.2 | g |
| Copper sulfate | 0.2 | g |
| Demineralized water qs | 100 | g |

EXAMPLE 4
Emulsion 3 (O/W)

| | | |
|---|---|---|
| Cyclomethicone | 10 | g |
| Cetyl dimethicone copolyol + polyglyceryl-4 | 3 | g |
| isostearate + hexyl laurate | | |
| Passionflower oil | 4 | g |
| Glycerol | 10 | g |
| PEG 12 | 10 | g |
| Magnesium aluminum silicate | 1.5 | g |
| Sucralfate | 3 | g |
| Copper sulfate | 0.3 | g |
| Zinc sulfate | 0.1 | g |
| Demineralized water qs | 100 | g |

EXAMPLE 5
Emulsion 4 (O/W)

| Sepigel 305 | 3.5 | g |
|---|---|---|
| Cyclomethicone | 6 | g |
| Propylene glycol | 5 | g |
| Xanthan gum | 0.2 | g |
| Triethanolamine qs | pH 6.5 | |
| Sucralfate | 0.5 | g |
| Copper sulfate | 0.1 | g |
| Zinc sulfate | 0.1 | g |
| Demineralized water qs | 100 | g |

EXAMPLE 6
Ointment

| | | |
|---|---|---|
| Petroleum jelly | 10 | g |
| Liquid paraffin | 8 | g |
| Beeswax | 4 | g |
| Isopropyl palmitate | 11 | g |
| Squalane | 5 | g |
| Ozokerite | 9 | g |
| Hydrogenated lanolin | 10 | g |
| Shea butter | 2 | g |
| Sucralfate | 1 | g |
| Zinc sulfate | 0.1 | g |
| Copper sulfate | 0.1 | g |
| Castor oil qs | 100 | g |

EXAMPLE 7
Pump-bottle vaporizer

| | | |
|---|---|---|
| Magnesium aluminum silicate | 5 | g |
| Sucralfate | 1 | g |
| Copper sulfate | 1 | g |
| Zinc sulfate | 1 | g |
| Avène water qs | 100 | g |

EXAMPLE 8
Aerosol powder spray

| | | |
|---|---|---|
| Micronized sucralfate | 2 | g |
| Copper sulfate | 0.2 | g |
| Zinc sulfate | 0.2 | g |
| Zinc oxide | 0.3 | g |
| Decamethylcyclosiloxane | 10 | g |
| Quaternium-18 hectorite | 1.2 | g |
| Propellent mixture (isobutane, propane, n-butane) qs (100 ml) | | |

Dermocosmetic Evaluation

The aim of this study was to evaluate the regenerating, cicatrizing and calmative properties of the cream of Example 1.

The experimental model adopted was the skin blister model. This is a standard technique generally used to comparatively study, on an untreated skin surface, the effect of a product on the rate and quality of re-epidermization of a fully delimited area of skin from which the epidermal layer above the dermo-epidermal function has been cut away beforehand.

Methodology

The test was performed on 6 volunteers. The experimental region selected is the inner face of the forearm (a region that is little exposed to ultraviolet radiation and to any external mechanical attack), on which six skin blisters with fully delimited contours were made.

After removing the detached epidermis, five blisters received the cream of Example 1 and/or the excipients of this cream (i.e. without the sucralfate, the copper sulfate and the zinc sulfate). The 6th blister was considered as the untreated control. The application of the excipient(s) was performed daily by a dermatologist for 14 consecutive days.

Each product was taken up using a disposable sterile syringe (without a needle) and then placed over the entire surface of the skin blister so as to form a uniform layer about 1 mm thick.

Each application was preceded by cleaning the blisters to be treated using sterile compresses impregnated with sterile physiological saline, by gentle vertical padding. Each blister was then covered with a sterile compress attached using an adhesive dressing. The dressings were left in place until the next clinical observation.

The regenerating properties were assessed by means of a quanti-qualitative method for measuring the rate and quality of epidermization over a 14-day period. The rate of epidermization was calculated (after measuring the injured areas by image analysis) according to the formula ST−S0/T with ST=area injured at time T and S0=area injured at time T0, T being the time in which a first total epidermization is obtained in a volunteer.

The quality of the epidermization was assessed by comparison of the clinical criteria relating to the quality of skin obtained during the controls at D14 and D1 (before the formation of the skin blisters).

The evaluation criteria were the following:
intensity of the erythema (1 mild erythema, 2 moderate erythema, 3 severe erythema)
thinness of the skin (0 very thin, 1 thin, 2 thick)
suppleness of the skin (0 not supple, 1 supple, 2 very supple)
normality of the epidermal regeneration (yes-no), given that an abnormal scar can be characterized by a hyper-hypotrophy or a hyper-hypopigmentation.
   Results
Rate of epidermization

The cream of Example 1 shows a mean rate of epidermization (Vi=11.72 mm²/day) that is twice as fast as that observed on an untreated blister and total reepidermization of 100% of the blisters on D4.

The excipient of this cream shows a mean rate of epidermization (V2=8 mm²/day) that is 1.5 times faster than that observed on untreated control blisters, and a reepidermization of 70% of the blisters at D4.
Evaluation of the quality of the epidermization between D1 and D4

Intensity of the Erythema

Cream of Example 1<excipient of Example 1<control

Thinness of the Skin

Cream of Example 1>excipient of Example 1>control

Suppleness of the Skin

Cream of Example 1>excipient of Example 1>control

Normal Epidermization (at D14)

Cream of Example 1>excipient of Example 1>control

The present invention thus also extends to the use of a combination of sucralfate and of copper and zinc sulfates in the amounts and proportions already mentioned above, for the manufacture of a dermocosmetic composition for a regenerative, cicatrizing and/or antiinflammatory treatment of the skin.

1. A cosmetic composition comprising a combination of sucralfate and of a mixture of copper and zinc sulfates, in an excipient allowing a topical application to the skin.

2. The cosmetic composition of claim 1, which contains from 0.01% to 5% by weight of sucralfate.

3. The cosmetic composition of claim 1, which contains 1% by weight of sucralfate.

4. The cosmetic composition of claims 1 which contains from 0.02% to 2% by weight of a mixture of copper and zinc sulfates.

5. The cosmetic composition of claim 1, which contains from 0.3% to 2% by weight of a mixture of copper and zinc sulfates.

6. The cosmetic composition of claim 1, wherein the weight ratio of sucralfate to the mixture of copper and zinc sulfates is between 0.5 and 20.

7. The cosmetic composition of claim 6, wherein the weight ratio of sucralfate to the mixture of copper and zinc sulfates is between 0.5 and 1.

8. The cosmetic composition of claim 1, wherein the weight ratio of copper sulfate to zinc sulfate is between 1 and 3.

9. A method for providing regenerative, cicatrizing and/or anti-inflammatory treatment of the skin of a living animal body comprising administering to the living animal body a composition of claim 1 which is effective therefore.
